(19) United States
(12) Patent Application Publication
(10) Pub. No.: US 2026/0123993 A1
(43) Pub. Date: May 7, 2026

(54) SYSTEMS AND METHODS FOR MACHINE-LEARNING-BASED OPTIMIZATION OF INTRAVASCULAR LITHOTRIPSY

(71) Applicant: Shockwave Medical, Inc., Santa Clara, CA (US)

(72) Inventors: Carlos H. LIMA, Campbell, CA (US); Peter Nabil COSTANDI, Brea, CA (US); Qing HE, Palo Alto, CA (US); Hadar CADOURI, San Jose, CA (US)

(73) Assignee: Shockwave Medical, Inc., Santa Clara, CA (US)

(21) Appl. No.: 18/939,908

(22) Filed: Nov. 7, 2024

(51) Int. Cl. A61B 34/10; A61B 17/00; A61B 17/22; A61B 18/26; G16H 10/60; G16H 20/40; G16H 40/63

(52) U.S. Cl. CPC A61B 34/10; A61B 17/22022; A61B 18/26; G16H 10/60; G16H 20/40; G16H 40/63; A61B 2017/00154; A61B 2017/22025; A61B 2018/263; A61B 2034/107

(57) ABSTRACT

Provided herein are methods and systems for treating a target area of a body lumen using an intravascular lithotripsy catheter, the method comprising: receiving, at a computing system, patient-specific information for a patient that has the at least partially occluded body lumen; determining, by the computing system, at least one parameter of a treatment of the at least partially occluded body lumen with the intravascular lithotripsy catheter, wherein the at least one parameter is determined by processing the patient-specific information with at least one machine learning model; and displaying, by the computing system, guidance for treating the at least partially occluded body lumen with the intravascular lithotripsy catheter based on the at least one parameter, wherein treating the at least partially occluded body lumen with the intravascular lithotripsy catheter comprises the intravascular lithotripsy catheter generating at least one shock wave.

### FIELD OF THE DISCLOSURE

The present disclosure relates generally to the field of medical devices and methods, and more specifically to shock wave catheter devices for treating calcified lesions in body lumens, such as calcified lesions and occlusions in vasculature and kidney stones in the urinary system.

### BACKGROUND

A wide variety of catheters have been developed for treating calcified lesions, such as calcified lesions in vasculature associated with arterial disease. For example, treatment systems for percutaneous coronary angioplasty or peripheral angioplasty use angioplasty balloons to dilate a calcified lesion and restore normal blood flow in a vessel. In these types of procedures, a catheter carrying a balloon is advanced into the vasculature along a guide wire until the balloon is aligned with calcified plaques. The balloon is then pressurized (normally to greater than 10 atm), causing the balloon to expand in a vessel to push calcified plaques back into the vessel wall and dilate occluded regions of vasculature.

More recently, the technique and treatment of intravascular lithotripsy (IVL) has been developed, which is an interventional procedure to modify calcified plaque in diseased arteries. The mechanism of plaque modification is through use of a catheter having one or more acoustic shock wave-generating sources located within a liquid that can generate acoustic shock waves that modify the calcified plaque. IVL devices vary in design with respect to the energy source used to generate the acoustic shock waves, with two exemplary energy sources being electrohydraulic generation and laser generation.

For electrohydraulic generation of acoustic shock waves, a conductive solution (e.g., saline) may be contained within an enclosure that surrounds electrodes or can be flushed through a tube that surrounds the electrodes. The calcified plaque modification is achieved by creating acoustic shock waves within the catheter by an electrical discharge across the electrodes. The energy from this electrical discharge enters the surrounding fluid faster than the speed of sound, generating an acoustic shock wave. In addition, the energy creates one or more rapidly expanding and collapsing vapor bubbles that generate secondary shock waves. The shock waves propagate radially outward and modify calcified plaque within the blood vessels. For laser generation of acoustic shock waves, a laser pulse is transmitted into and absorbed by a fluid within the catheter. This absorption process rapidly heats and vaporizes the fluid, thereby generating the rapidly expanding and collapsing vapor bubble, as well as the acoustic shock waves that propagate outward and modify the calcified plaque. The acoustic shock wave intensity is higher if a fluid is chosen that exhibits strong absorption at the laser wavelength that is employed. These examples of IVL devices are not intended to be a comprehensive list of potential energy sources to create IVL shock waves.

The IVL process may be considered different from standard atherectomy procedures in that it cracks calcium but does not liberate the cracked calcium from the tissue. Hence, generally speaking, IVL should not require aspiration nor embolic protection. Further, due to the compliance of a normal blood vessel and non-calcified plaque, the shock waves produced by IVL do not modify the normal vessel tissue or non-calcified plaque. Moreover, IVL does not carry the same degree of risk of perforation, dissection, or other damage to vasculature as atherectomy procedures or angioplasty procedures using cutting or scoring balloons.

More specifically, catheters to deliver IVL therapy have been developed that include pairs of electrodes for electrohydraulically generating shock waves inside an angioplasty balloon. Shock wave devices can be particularly effective for treating calcified plaque lesions because the acoustic pressure from the shock waves can crack and disrupt lesions near the angioplasty balloon without harming the surrounding tissue. In these devices, the catheter is advanced over a guidewire through a patient's vasculature until it is positioned proximal to and/or aligned with a calcified plaque lesion in a body lumen. The balloon is then inflated with conductive fluid (using a relatively low pressure of 1-4 atm) so that the balloon expands to contact the lesion but is not an inflation pressure that substantively displaces the lesion. Voltage pulses can then be applied across the electrodes of the electrode pairs to produce acoustic shock waves that propagate through the walls of the angioplasty balloon and into the lesions. Once the lesions have been cracked by the acoustic shock waves, the balloon can be expanded further to increase the cross-sectional area of the lumen and improve blood flow through the lumen. Alternative devices to deliver IVL therapy can be within a closed volume other than an angioplasty balloon, such as a cap, balloons of variable compliancy, or other enclosure.

Different shock wave device configurations may be available for different applications. For example, larger shock wave devices may have a greater number of shock wave emitters and/or may be able to generate greater shock wave energy, which may be ideal for treating occlusions in larger vasculature, whereas smaller shock wave devices may be capable of traversing smaller vasculature. Shock wave devices having forward-biased and/or forward-directed shock wave emitters may be suitable for treatment of difficult-to-cross occlusions, whereas shock wave devices with multiple spaced-apart radially directed shock wave emitters may be suitable for treating occlusions that are annular and relatively extensive. Similarly, the same shock wave emitter can be used in different ways depending on the treatment. Some occlusions may be more effectively treated with a greater number of lower-amplitude shock wave pulses, whereas other occlusions may respond better to fewer, higher-amplitude pulses. The selection of the appropriate shock wave device and/or the manner in which a shock wave device is used for a given treatment is often based on the training and experience of the surgeon, which can lead to variability in the effectiveness of treatment.

### SUMMARY

According to various aspects, systems and methods include using one or more machine learning models to determine one or more parameters associated with treatment of a target area of a body lumen with a lithotripsy catheter. The one or more parameters can include parameters for guiding a treatment provider for selecting a suitable catheter for a treatment, for guiding the treatment provider in operating the catheter for the treatment, and/or for controlling one or more aspects of operation of the catheter. The one or more machine learning models may be configured to process patient-specific information for determining one or more parameters that optimize or otherwise tailor the treatment to the patient. The systems and methods described herein may assist treatment providers in achieving improved and/or more consistent patient outcomes.

In some examples, a method for treating a target area of a body lumen using an intravascular lithotripsy catheter is provided, the method comprising: receiving, at a computing system, patient-specific information for a patient that has the at least partially occluded body lumen; determining, by the computing system, at least one parameter of a treatment of the at least partially occluded body lumen with the intravascular lithotripsy catheter, wherein the at least one parameter is determined by processing the patient-specific information with at least one machine learning model; and displaying, by the computing system, guidance for treating the at least partially occluded body lumen with the intravascular lithotripsy catheter based on the at least one parameter, wherein treating the at least partially occluded body lumen with the intravascular lithotripsy catheter comprises the intravascular lithotripsy catheter generating at least one shock wave.

In some examples, the patient-specific information comprises imaging data capturing the at least partially occluded body lumen. In some examples, the patient-specific information comprises measurements of the body lumen and/or an occlusion in the body lumen. In some examples, at least one parameter comprises a type of the intravascular lithotripsy catheter or a size of the intravascular lithotripsy catheter. In some examples, the at least one parameter comprises at least one parameter for operating the intravascular lithotripsy catheter. In some examples, the at least one parameter for operating the intravascular lithotripsy catheter comprises a fluid filling pressure of an enclosure of the intravascular lithotripsy catheter, or at least one parameter of at least one pulse of energy provided to the intravascular lithotripsy catheter for generating shock waves. In some examples, the at least one parameter of the at least one pulse of energy comprises a pulse frequency, a pulse amplitude, and a total number of pulses. In some examples, the at least one parameter comprises a number of cycles of pulses. In some examples, the at least one pulse of energy is at least one electrical pulse or at least one laser pulse.

In some examples, a system for treating a target area of a body lumen using an intravascular lithotripsy catheter is provided, the system comprising one or more processors and memory storing one or more programs for execution by the one or more processors, the one or more programs including instructions that when executed by the one or more processors cause the system to perform the method of any of the aforementioned examples.

In some examples, a method for treating a target area of a body lumen using an intravascular lithotripsy catheter is provided, the method comprising: receiving, at a computing system, data generated during treatment of the at least partially occluded body lumen by the intravascular lithotripsy catheter; determining, by the computing system, at least one parameter for operating the intravascular lithotripsy catheter by processing the data generated during the treatment of the at least partially occluded body lumen with at least one machine learning model; and providing guidance to a treatment provider for operating the intravascular lithotripsy catheter in accordance with the at least one parameter for operating the intravascular lithotripsy catheter.

In some examples, the data generated during the treatment of the at least partially occluded body lumen by the intravascular lithotripsy catheter comprises imaging data capturing the at least partially occluded body lumen. In some examples, the data generated during the treatment of the at least partially occluded body lumen by the intravascular lithotripsy catheter comprises measurements of the body lumen and/or an occlusion in the body lumen. In some examples, the at least one parameter comprises at least one parameter for operating the intravascular lithotripsy catheter. In some examples, the at least one parameter for operating the intravascular lithotripsy catheter comprises a fluid filling pressure of an enclosure of the intravascular lithotripsy catheter, or at least one parameter of at least one pulse of energy provided to the intravascular lithotripsy catheter for generating shock waves. In some examples, the at least one parameter of the at least one pulse of energy comprises a pulse frequency, a pulse amplitude, and a total number of pulses. In some examples, the at least one parameter comprises a number of cycles of pulses. In some examples, the at least one pulse of energy is at least one electrical pulse or at least one laser pulse. In some examples, the at least one parameter for operating the intravascular lithotripsy catheter is determined by processing patient-specific data received by the computing system prior to the treatment.

In some examples, a system for treating a target area of a body lumen using an intravascular lithotripsy catheter is provided, the system comprising one or more processors and memory storing one or more programs for execution by the one or more processors, the one or more programs including instructions that when executed by the one or more processors cause the system to perform the method of any of the aforementioned examples.

In some examples, a method for treating a target area of a body lumen using an intravascular lithotripsy catheter is provided, the method comprising: receiving, at a computing system, data generated during treatment of the at least partially occluded body lumen by the intravascular lithotripsy catheter; determining, by the computing system, at least one parameter for operating the intravascular lithotripsy catheter by processing the data associated with treatment of the occlusion with at least one machine learning model; and automatically controlling treatment by the intravascular lithotripsy catheter according to the at least one parameter to treat the occlusion.

In some examples, the at least one parameter comprises a fluid filling pressure of an enclosure of the intravascular lithotripsy catheter or at least one parameter of at least one pulse of energy provided to the intravascular lithotripsy catheter for generating shock waves. In some examples, the at least one parameter of the at least one pulse of energy comprises a pulse frequency, a pulse amplitude, and a total number of pulses. In some examples, the data generated during the treatment of the occlusion comprises imaging data capturing the at least partially occluded body lumen. In some examples, automatically controlling treatment by the intravascular lithotripsy catheter according to the at least one parameter comprises automatically controlling, by the computing system, an energy pulse generator that provides energy pulses to the intravascular lithotripsy catheter for generating the at least one shock wave. In some examples, automatically controlling treatment by the intravascular lithotripsy catheter according to the at least one parameter comprises automatically controlling, by the computing system, a pump to control a fluid pressure within a fluid enclosure of the intravascular lithotripsy catheter. In some examples, the intravascular lithotripsy catheter comprises a fluid filled enclosure within which the at least one shock wave is generated, and wherein the at least one parameter comprises a proportion of contrast in a fluid that fills the fluid filled enclosure. In some examples, automatically controlling treatment by the intravascular lithotripsy catheter according to the at least one parameter comprises automatically controlling, by the computing system, at least one valve for controlling the proportion of contrast in the fluid. In some examples, the at least one parameter for operating the intravascular lithotripsy catheter is determined by processing patient-specific data received by the computing system prior to the treatment.

In some examples, a system for treating a target area of a body lumen using an intravascular lithotripsy catheter is provided, the system comprising one or more processors and memory storing one or more programs for execution by the one or more processors, the one or more programs including instructions that when executed by the one or more processors cause the system to perform the method of any of the aforementioned examples.

### DESCRIPTION OF THE FIGURES

The invention will now be described, by way of example only, with reference to the accompanying drawings, in which:

FIG. 1 illustrates an exemplary system for treating lesions in body lumens;

FIG. 2 illustrates an exemplary method for training a machine learning model for use in determining at least one parameter of a treatment of a target area of a body lumen with a lithotripsy catheter;

FIG. 3 illustrates an exemplary method for treating a target area of a body lumen using a lithotripsy catheter in which a machine learning model is used to provide guidance for the treatment;

FIG. 4 illustrates an exemplary treatment system configured for obtaining treatment data and using the treatment data for providing updated guidance during lithotripsy treatment;

FIG. 5 is a functional block diagram of an exemplary computing system configured to pre-process treatment data and generate updated guidance;

FIG. 6 illustrates an exemplary method for automatically controlling treatment of a target area of a body lumen using an intravascular lithotripsy catheter;

FIG. 7 illustrates an exemplary arrangement for automatically controlling the operation of a fluid supply system for supplying fluid to a lithotripsy catheter during lithotripsy treatment; and

FIG. 8 illustrates an exemplary computing system.

### DETAILED DESCRIPTION

The following description is presented to enable a person of ordinary skill in the art to make and use the various embodiments and aspects thereof disclosed herein. Descriptions of specific devices, assemblies, techniques, and applications are provided only as examples. Various modifications to the examples described herein will be readily apparent to those of ordinary skill in the art, and the general principles described herein may be applied to other examples and applications without departing from the spirit and scope of the various embodiments and aspects thereof. Thus, the various embodiments and aspects thereof are not intended to be limited to the examples described herein and shown but are to be accorded the scope consistent with the claims.

Described herein are systems and methods that utilize one or more machine learning models for determining one or more parameters associated with treatment of a target area (e.g., a calcified lesion or occlusion) of a body lumen with a lithotripsy catheter. The one or more machine learning models are configured to process patient-specific information (optionally, along with device information) for generating treatment-related parameters that are optimized, or otherwise tailored, for the patient. The treatment-related parameter(s) may be used to provide guidance to a treatment provider (e.g., a surgeon), such as guidance for selecting a suitable catheter for a treatment, may be used for generation of training simulations pre-operatively, or may be used for other purposes. The treatment-related parameter(s) may be used to provide guidance on operating a catheter. For example, one or more parameters for operation of an energy pulse generator that provide energy pulses to a treatment catheter for generating shock waves and/or one or more parameters for operation of a fluid supply system that provides fluid to the catheter may be provided to a treatment provider to guide the treatment provider in operating the energy pulse generator and/or fluid supply system. For example, the at least one parameter can be a mode that the treatment provider should select for operating the energy pulse generator and/or fluid supply system or a specific setting of the energy pulse generator and/or fluid supply system, such as a setting of the energy pulse generator associated with pulse amplitude or pulse frequency or a setting of the fluid supply system associated with the pressure of fluid within an enclosure of the catheter. Additionally, or alternatively, the treatment-related parameter(s) may be used to automatically control one or more aspects of operation of the catheter during treatment, such as pulse amplitude or pulse frequency or a pressure of fluid within an enclosure of the catheter.

Patient-specific information that may be processed by one or more machine learning models for determining treatment-related parameters can include information about the body lumen and/or lesion. Such information can include measurements or other characteristics associated with the body lumen (diameter(s), location(s), type, etc.) and/or measurements or other characteristics associated with the lesion (lengths, widths, thicknesses, hardness, location, eccentricity, degree of plaque burden, etc.). Geometric characteristics of a lesion may be used, for example, with a finite-element model to calculate target locations for shock wave therapy. In some embodiments, such target locations of a lesion include parts of the lesion where geometry changes abruptly (e.g., a corner or an edge). In some embodiments, shock wave emitters are positioned at calculated target locations for shock wave therapy. Patient-specific information can include patient demographic information (e.g., age, sex, etc.), patient monitoring data (e.g., heart rate, blood pressure, etc.), patient scans (e.g., angiograms), information about pre-existing conditions and/or medications, or any other data associated with the patient that may be relevant to treating the lesion.

Machine learning models for determining treatment-related parameters can be trained using a supervisory learning process on historical treatment data. The historical treatment data can be any data associated with previously performed treatments of target areas in body lumens with lithotripsy catheters (historical treatment data should be anonymized to adhere to patient privacy requirements). Examples of historical treatment data include pre-, intra-, and post-operative scans (such as CT scans, angiograms, IVUS scans, OCT scans, and MRI scans), pre-treatment and/or post-treatment characteristics of lumens and lesions, objective and/or subjective treatment outcome assessments, and/or post-treatment or intra-treatment complications (e.g., restenosis, vessel collapse, thrombus formation). The historical treatment data can include data associated with performance of treatments, such as information about the type, size, or other characteristics of a catheter used during treatment and/or data associated with operation of an energy pulse generator and/or fluid supply system (e.g., fluid salinity, contrast agent concentration, inflation pressure). Other types of historical treatment data may include voltage pulse generator logs. Other types of historical treatment data may include simulated data. Other types of historical treatment may include animal study data. The historical treatment data may be labeled to generate training data used for training the machine learning model(s).

Systems and methods described herein can assist treatment providers in achieving improved and/or more consistent patient outcomes. Treatments can be optimized for each patient, leading to improved treatment outcomes. By utilizing machine learning models trained on historical treatment data, the amount of training and experience needed for providing successful treatment can be reduced, potentially increasing the availability of treatment and reducing treatment cost. Additionally, or alternatively, optimization of treatment with machine learning can reduce procedure time (e.g., by reducing the amount of trial and error), which may lead to an increase in the number of procedures that can be performed by a given surgeon or surgical team or that can be done in a given operating room. Reduction of treatment time may be most impactful for treatments involving complex lesions where even an experienced treatment providers may struggle to determine the optimal treatment approach. Optimization of treatment with machine learning may have the benefit of increasing lithotripsy catheter utilization, such as by not over-using (e.g., providing more pulses than needed and/or by providing higher amplitude pulses than needed) a catheter for a given treatment, thereby increasing the number of treatments for which a catheter may be used. As used herein, the term “electrode” refers to an electrically conducting element (typically made of metal) that receives electrical current and subsequently releases the electrical current to another electrically conducting element. In the context of the present disclosure, electrodes are often positioned relative to each other, such as in an arrangement of an inner electrode and an outer electrode. Accordingly, as used herein, the term “electrode pair” refers to two electrodes that are positioned adjacent to each other such that application of a sufficiently high voltage to the electrode pair will cause an electrical current to transmit across the gap (also referred to as a “spark gap”) between the two electrodes (e.g., from an inner electrode to an outer electrode, or vice versa, optionally with the electricity passing through a conductive fluid or gas therebetween). In some contexts, one or more electrode pairs may also be referred to as an electrode assembly. In the context of the present disclosure, the term “emitter” broadly refers to the region of an electrode assembly where the current transmits across the electrode pair, generating a shock wave. The terms “emitter sheath” and “emitter band” refer to a continuous or discontinuous band of conductive material that may form one or more electrodes of one or more electrode pairs, thereby forming a location of one or more emitters.

Components of emitters, including electrodes and emitter sheaths/bands, may be formed from a metal, such as stainless steel, copper, tungsten, platinum, palladium, molybdenum, cobalt, chromium, iridium, an alloy or alloys thereof, such as cobalt-chromium, platinum-chromium, cobalt-chromium-platinum-palladium-iridium, or platinum-iridium, or a mixture of such materials.

For treatment of an occlusion in a blood vessel, the voltage pulse applied by a power source, including any of the power sources described herein (which may also be referred to herein as voltage sources or pulse generators), is typically in the range of from about five hundred to fifteen thousand volts (500 V-15,000 V). In some implementations, the voltage pulse applied by the voltage source can be up to about fifteen thousand volts (15,000 V) or higher than fifteen thousand volts (15,000 V). The pulse width of the applied voltage pulses ranges between two microseconds and six microseconds (2-100 μs). The repetition rate or frequency of the applied voltage pulses may be between about 1 Hz and 100 Hz. The total number of pulses applied by the power source may be, for example, sixty (60) pulses, eighty (80) pulses, one hundred twenty (120) pulses, three hundred (300) pulses, or up to five hundred (500) pulses, or any increments of pulses within this range. Alternatively, or additionally, in some examples, the power source may be configured to deliver a packet of micro-pulses having a sub-frequency between about one hundred hertz to ten kilohertz (100 Hz-10 kHz). The preferred voltage, repetition rate, and number of pulses may vary depending on, e.g., the size of the lesion, the extent of calcification, the size of the blood vessel, the attributes of the patient, or the stage of treatment. For instance, a physician may start with low energy shock waves and increase the energy as needed during the procedure, or vice versa. The magnitude of the shock waves can be controlled by controlling the voltage, current, duration, and repetition rate of the pulsed voltage from the power source.

In some embodiments, an IVL catheter is a so-called “rapid exchange-type” (“Rx”) catheter provided with an opening portion through which a guide wire is guided (e.g., through a middle portion of a central tube in a longitudinal direction). In other embodiments, an IVL catheter may be an “over-the-wire-type” (“OTW”) catheter in which a guide wire lumen is formed throughout the overall length of the catheter, and a guide wire is guided through the proximal end of a hub.

Although shock wave devices described herein generate shock waves based on high voltage applied to electrodes, it should be understood that a shock wave device additionally or alternatively may comprise a laser and optical fibers as a shock wave emitter system whereby the laser source delivers energy through an optical fiber and into a fluid to form shock waves and/or cavitation bubbles.

In the following description of the various embodiments, reference is made to the accompanying drawings, in which are shown, by way of illustration, specific embodiments that can be practiced. It is to be understood that other embodiments and examples can be practiced, and changes can be made without departing from the scope of the disclosure.

In addition, it is also to be understood that the singular forms “a,” “an,” and “the” used in the following description are intended to include the plural forms as well, unless the context clearly indicates otherwise. It is also to be understood that the term “and/or” as used herein refers to and encompasses any and all possible combinations of one or more of the associated listed items. It is further to be understood that the terms “includes, “including,” “comprises,” and/or “comprising,” when used herein, specify the presence of stated features, integers, steps, operations, elements, components, and/or units but do not preclude the presence or addition of one or more other features, integers, steps, operations, elements, components, units, and/or groups thereof. As provided herein, it should be appreciated that any disclosure of a numerical range describing dimensions or measurements such as thicknesses, length, weight, time, frequency, temperature, voltage, current, angle, etc. is inclusive of any numerical increment or gradation within the ranges set forth relative to the given dimension or measurement. Furthermore, numerical designators such as “first,” “second,” “third,” “fourth,” etc. are merely descriptive and do not indicate a relative order, location, or identity of elements or features described by the designators. For instance, a “first” shock wave may be immediately succeeded by a “third” shock wave, which is then succeeded by a “second” shock wave. As another example, a “third” emitter may be used to generate a “first” shock wave and vice versa. Accordingly, numerical designators of various elements and features are not intended to limit the disclosure and may be modified and interchanged without departing from the subject invention.

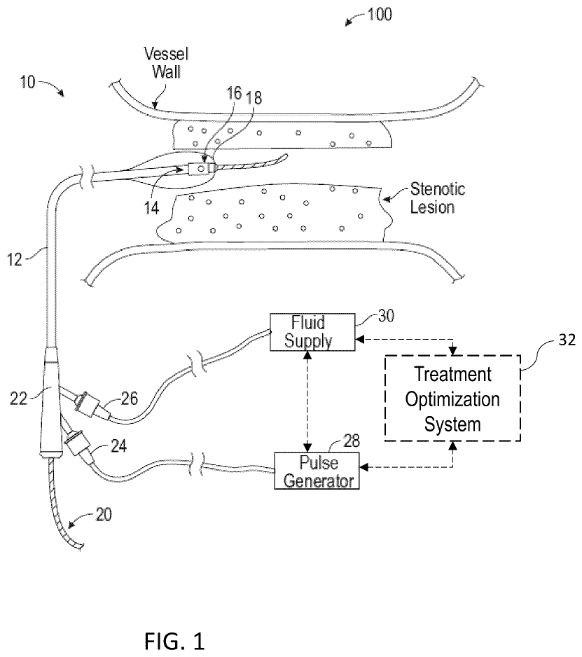

FIG. 1 illustrates a system 100 for treating lesions, such as calcifications and fibrous tissue, in body lumens. The system includes a lithotripsy catheter 10 configured for generating shock waves. The catheter 10 may be used to treat a lesion within a body lumen, such as by fragmenting, cracking, or otherwise breaking up calculi of the lesion, for instance, to treat various occlusions within vasculature of a patient. The catheter 10 includes at least one shock wave emitter 16 positioned within an enclosure 18. In general, the catheter 10 is advanced to a target lesion in a body lumen of a patient, such as the stenotic lesion in the vessel depicted in FIG. 1, and the catheter 10 is operated to generate shock waves that treat the lesion. The catheter 10 may be advanced over a guidewire 20 carried in a guidewire lumen of the catheter 10. Alternatively, the catheter 10 is advanced without the use of a guidewire.

The catheter 10 can include any number of shock wave emitters 16. A shock wave emitter 16 may include electrode pairs having first and second electrodes separated by a gap at which shock waves are formed when a current flows across the gap between the electrodes of the pair (i.e., when a voltage is applied across the first and second electrodes). Electrode pairs may be formed by an emitter band and a plurality of conductors positioned adjacent to the emitter band. Each conductor, together with the emitter band, may define a respective electrode pair. A shock wave emitter 16 may be configured to generate shock waves using laser energy. For examples, the shock wave emitter 16 may be provided by a distal end of an optical fiber such that when laser pulses are directed to the optical fiber, the laser pulses are emitted from the optical fiber into surrounding fluid to generate one or more shock waves.

The catheter 10 may include a flexible shaft 12 that extends from a proximal end 22 of the catheter 10 to a distal end 14 of the catheter 10. The shaft 12 provides various internal conduits connecting elements of the distal end 14 with the proximal end 22 of the catheter 10. The shaft 12 may include an elongate tube that includes a lumen for receiving the guidewire 20. The elongate tube may include one or more additional lumens, such as for carrying fluid to and/or receiving fluid from the distal end 14.

An enclosure 18 (e.g., a low-profile flexible angioplasty balloon, a polymer membrane in tension that can flex outward, etc.) may be positioned proximate to the distal end 14 of the catheter 10, forming an annular channel around the shaft 12 of the catheter 10. The enclosure may be connected at one or both of its ends to the shaft 12 and may be sealed at one or both ends to the shaft 12. The enclosure 18 surrounds the shock wave emitter(s) 16, such that shock waves are produced within the enclosure 18. The enclosure 18 may be filled or inflated with a fluid, such as a conductive fluid (e.g., saline). The fluid allows the shock waves to propagate outwardly from the shock wave emitter(s) 16 through the walls of the enclosure 18 and then into the target lesion. In one or more examples, the fluid may include an agent configured to absorb laser energy to generate shock waves. The fluid may include contrast fluid to enable fluoroscopic viewing of the catheter 10 during use. In some implementations, the material that forms the primary surface(s) of the enclosure 18 through which shock waves pass can be a compliant or semi-compliant polymer. A compliant enclosure, such as an angioplasty balloon, may be inflated to provide pressure to surrounding tissue of a body lumen to expand the body lumen. In other implementations, enclosure 18 may be a rigid and inflexible structure, which may provide the advantage of a relatively small crossing profile. The enclosure 18 may mitigate thermal injury to soft tissue and reduce cavitation stresses by limiting expansion of vapor bubbles that may be produced during shock wave generation to the interior of the enclosure. For instance, the vapor bubbles may hit the enclosure wall before reaching their maximum potential size, thus inducing collapse, and reducing cavitation stress and preventing soft tissue injury that can be caused by tensile stresses during cavitation bubble collapse.

The catheter 10 includes a proximal end 22 that remains outside of a patient's vasculature during treatment. The catheter 10 may include an entry port at the proximal end 22 for receiving the guidewire 20. The catheter 10 may include a fluid port 26 at the proximal end 22 for receiving a fluid from a fluid supply system 30 for filling and emptying the enclosure 18 during use of the catheter 10. The catheter 10 may include a pulse delivery port 24 at the proximal end 22 to provide energy pulses to the shock wave emitter(s) 16. The energy pulses may be generated by an energy pulse generator 28. The energy pulse generator 28 may be configured for generating electrical pulses for providing to one or more shock wave emitters 16 comprised of electrodes or may be configured for generating laser pulses for providing to one or more shock wave emitters 16 comprised of one or more optical fibers.

The energy pulse generator 28 is configured to provide energy pulses to the one or more shock wave emitters 16 according to one or more adjustable parameters such that shock waves generated by the shock wave emitter(s) 16 have different characteristics depending on the parameters used. Exemplary adjustable parameters of the energy pulses include, but are not limited to, voltage used for generating pulses, pulse amplitude, pulse frequency, pulse repetition rate, pulse width, number of pulses per cycle, number of cycles per treatment or per treatment stage, which of multiple shock wave emitters are used for generating shock waves and their pattern of usage, etc. The energy pulse generator 28 may operate in different modes associated with different settings of one or more adjustable parameters. For example, one mode that is associated with relatively high shock wave intensity may have relatively high pulse amplitude and/or frequency, and another mode that is associated with relatively low shock wave intensity (relative to the high intensity mode) may have relatively low pulse amplitude and/or frequency. The different modes may be used for different stages of a treatment, different anatomy, and/or different types or sizes of catheter 10. Optionally, one or more adjustable parameters may be adjusted while the catheter 10 is in operation—i.e., while delivering pulses. The energy pulse generator 28 may be communicatively connected to the fluid supply system 30, such as for controlling one or more aspects of the operation of the fluid supply system 30.

The fluid supply system 30 may be configured to provide fluid to the catheter 10 according to one or more adjustable parameters. Exemplary adjustable parameters include, but are not limited to, fluid pressure, static and/or dynamic fluid pressure profile, fluid flow rate (e.g., in examples in which a catheter 10 that has fluid flow capability), activation/deactivation of different fluid flow paths (e.g., an aspiration flow path, an enclosure pressurization flow path, etc.), and/or a mixture ratio of contrast with a base fluid such as saline. The fluid supply system 30 may operate in different modes associated with different settings of one or more adjustable parameters. For example, a first mode may be configured to provide relatively high-pressure fluid to enclosure 18 to expand the enclosure 18 for contacting and applying pressure to the walls of a body lumen, while a second mode may be configured to provide low-pressure fluid to the enclosure 18 that is intended to fill the enclosure 18 with fluid but not expand it. The different modes may be used for different stages of treatment, different anatomy, and/or different types or sizes of catheter 10. Optionally, one or more adjustable parameters may be adjusted while the catheter 10 is in use, which may include parameter adjustment while generating shock waves.

System 100 may include a treatment optimization system 32 configured for optimizing treatment of a lesion by the catheter 10 based on one or more characteristics of the treatment, such as based on one or more patient characteristics, one or more body lumen characteristics, one or more lesion characteristics, etc. Treatment optimization system 32 may provide for treatment optimization in a number of ways. Treatment optimization system 32 may provide guidance to a treatment provider, such as guidance for selecting a suitable catheter type and/or size and/or guidance for operating one or more of the catheter 10, the energy pulse generator 28, and/or the fluid supply system 30. Optionally, treatment optimization system 32 may provide guidance on an optimal location to position one or more emitters. For example, there may be locations on a lesion (like corners or edges) where stress can concentrate and lead to cracks, and treatment optimization system 32 may identify such locations and may indicate such locations to a treatment provider (e.g., may provide a graphical indication on an image of the lumen indicating the location(s) where the treatment provider should position one or more emitters). Treatment optimization system 32 may provide operational parameters to the energy pulse generator 28 and/or the fluid supply system 30. Treatment optimization system 32 may directly control one or more aspects of operation of one or more of the catheter 10, the energy pulse generator 28, and/or the fluid supply system 30.

In general, treatment optimization system 32 includes one or more processors and memory storing one or more programs for execution by the one or more processors for providing the functionality of treatment optimization system 32 described herein. Treatment optimization system 32 may be communicatively connected to the energy pulse generator 28 and/or the fluid supply system 30 via one or more communication connections (wired and/or wireless), which may include one or more communication networks used to connect treatment optimization system 32 to the energy pulse generator 28 and/or the fluid supply system 30. Treatment optimization system 32 may include both local and remote components. For example, a component of treatment optimization system 32 that is local to a treatment location and communicates directly with the fluid supply system 30 and/or the energy pulse generator 28 may utilize data storage and/or processing capabilities of a remote component of treatment optimization system 32, which may be hosted in a dedicated server or in the cloud.

Treatment optimization system 32 (or a local component of treatment optimization system 32) may be communicatively connected to a controller of the energy pulse generator 28 and/or a controller of the fluid supply system 30. Alternatively, treatment optimization system 32 may be a component or sub-system of the energy pulse generator 28 or the fluid supply system 30. Treatment optimization system 32 may receive information from one or more of the catheter 10, the energy pulse generator 28, and/or the fluid supply system 30. For example, treatment optimization system 32 may receive operating parameter settings from pulse generator 28 and/or fluid supply system 30 and/or may receive sensor data from pulse generator 28, fluid supply system 30, and/or catheter 10. Treatment optimization system 32 may send information, such as operating parameter settings and/or control commands, to the energy pulse generator 28 and/or the fluid supply system 30 for controlling operation of the energy pulse generator 28 and/or the fluid supply system 30.

Treatment optimization system 32 may be configured to optimize treatment by determining one or more treatment-related parameters based on one or more characteristics associated with a planned treatment and/or an ongoing treatment. The one or more treatment parameters can include, for example, parameters associated with the configuration of the catheter 10, such as catheter type, catheter size, etc. Different catheter types or sizes may be available for use in different-sized body lumens, may have different numbers and/or arrangements of shock wave emitters, or may have different enclosure styles and/or sizes. Treatment optimization system 32 may be configured to determine the optimal catheter configuration for use in a given treatment based on the characteristics associated with the treatment, such as the body lumen type and/or size, lesion size or shape, lesion location, etc.

The one or more treatment parameters that may be determined by treatment optimization system 32 can include one or more parameters associated with use of the catheter 10. Such parameters may include parameters associated with operation of the fluid supply system 30, such as fluid pressure of the fluid supplied to the enclosure 18 by the fluid supply system 30, a dynamic fluid pressure profile, or a mixture of fluid constituents (e.g., a proportion of contrast to saline). Parameters associated with use of the catheter 10 can include one or more parameters association with operation of the energy pulse generator 28, such as pulse amplitude, pulse frequency, number of pulses per cycle, and/or number of cycles of the energy pulses provided by the energy pulse generator 28.

The one or more treatment parameters can be associated with phases of a treatment, such as how long the treatment provider should apply shock waves to a lesion before pressurizing the enclosure 18 to expand the body lumen and/or how long to expand the body lumen with the enclosure 18. The one or more treatment parameters can be associated with post-lithotripsy steps, such as whether or not to perform angioplasty or whether or not to implant a stent.

Treatment optimization system 32 may be configured to determine one or more treatment parameters based on patient-specific information. Patient-specific information can include characteristics of a body lumen and/or lesion obtained from one or more scans of the patient, such as CT scans, angiograms, intravascular ultrasound (IVUS) scans, optical coherence tomography (OCT) scans, or MRI scans. Characteristics of a body lumen can include, for example, body lumen length, diameter at one or more locations of the lumen, and lumen taper. Characteristics of a lesion can include lesion size, lesion shape, percentage of occlusion of the lumen by the lesion, and lesion hardness.

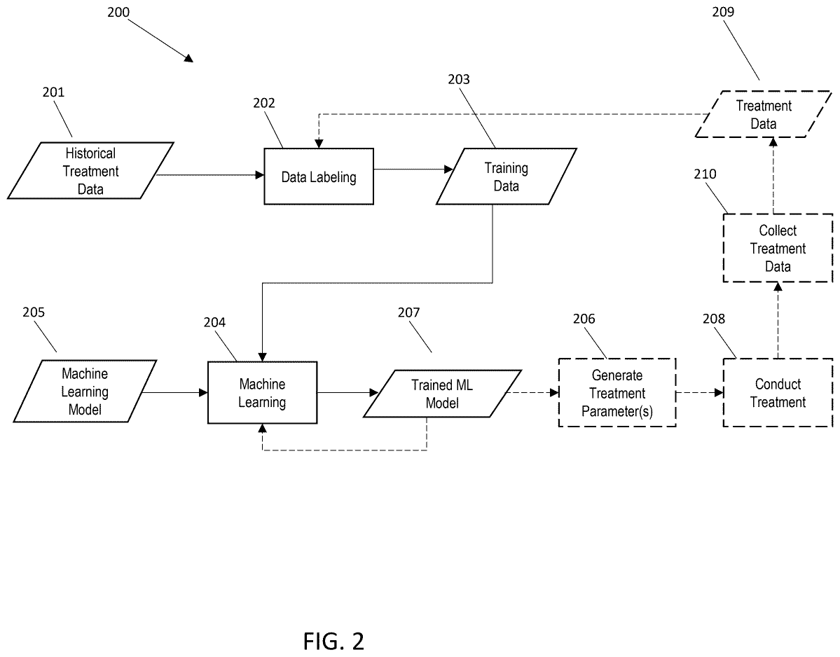

Treatment optimization system 32 may be configured to determine one or more treatment parameters using one or more machine learning models configured to process patient-specific information to determine treatment parameters optimized for, or otherwise tailored to, a patient. The one or more machine learning models are trained on data associated with previously performed treatments. FIG. 2 illustrates an exemplary method 200 for training a machine learning model for use in determining at least one parameter of a treatment of a target area of a body lumen with a lithotripsy catheter, such as catheter 10 of FIG. 1. Method 200 is performed by one or more computing systems.

At step 202, historical treatment data 201 is labeled to create training data 203. The historical treatment data can be any data associated with previously performed treatments of target treatment areas of body lumens with lithotripsy catheters. Historical treatment data may be anonymized to adhere to patient privacy requirements. Historical treatment data can include pre-, intra-, and post-operative scans (such as CT scans, angiograms, IVUS scans, OCT scans, and MRI scans). Historical treatment data can include patient attributes, such as age, sex, weight, height, and health history. Historical treatment data can include characteristics of lumens, such as length, diameter(s), tapering characteristics (such as direction of taper and diameter ratios), and/or type of lumen (such as coronary vs. peripheral vasculature). Historical treatment data can include characteristics of treated lesions, such as lesion makeup (e.g., calcifications, fibrous tissue, eccentricity, etc.), lesion hardness, lesion size (absolute and/or relative to the lumen size), and/or degree of occlusion of the lumen. Historical treatment data can include pre-treatment characteristics and/or post-treatment characteristics. Historical treatment data can include objective and/or subjective treatment outcome assessments and/or post-treatment or intra-treatment complications (e.g., restenosis, vessel collapse, thrombus formation, long-term survivability, etc.).

Historical treatment data can include data associated with the performance of treatment. Such historical treatment data can include information about a catheter used during treatment, such as information associated with a catheter size or configuration. Historical treatment data can include data associated with operation of an energy pulse generator, such as pulse generator 28 of FIG. 1. The data associated with the operation of an energy pulse generator can include any of the energy pulse generator attributes described herein and any other attributes for a given pulse generator. Such attributes can include the number of pulses (total and/or per cycle or stage of treatment), number of cycles of pulses, pulse amplitudes, and pulse frequencies. Historical treatment data can include data associated with operation of a fluid supply system, such as fluid supply system 30 of FIG. 1. Data associated with operation of a fluid supply system may include the type or other attribute(s) of fluid used to fill the enclosure of the catheter surrounding the shock wave emitters (e.g., enclosure 18 of FIG. 1), the pressure of the fluid of the enclosure, or the pressure profile over the span of a treatment. Historical treatment data can include sensor data collected during a treatment. The sensor data can include, for example, data from a pressure sensor associated with the pressure within enclosure 18, temperature data from a temperature sensor within or proximate the enclosure 18, voltage data from a voltage sensor electrically connected to the shock wave emitter(s), or current data from a current sensor electrically connected to the shock wave emitter(s). Historical treatment data can include assessment of imaging data.

Historical treatment data can be obtained from multiple different sources. Historical treatment data can be obtained from one or more treatment centers (e.g., hospitals, out-patient clinics, etc.), from one or more research centers, from one or more clinical data storage systems, or from any other source. Again, data of this sort should be anonymized for patient privacy.

Other kinds of training data that may be used for training a machine learning model include finite element analysis and bench top models showing trends for the stress and number of fractures required for different lesion characteristics (e.g., shape, hardness, etc.).

Labeling of the treatment data in step 202 can include trained personnel associating treatment data with one or more outcome assessments. For example, the treatment data associated with a given procedure may be given a binary label associated with a “good” outcome or a “bad” outcome according to the labeler's assessment of the outcome of the treatment. More complex labeling schemes can be used, including labeling schemes associated with “scoring” treatments based on one or more treatment success criteria, such as whether further treatments were needed, presence or prevalence of side effects, degree of recovery, or any other treatment success criteria. Labeling schemes may include objective measurements associated with treatment outcomes, such as percent occlusion reduction, percent lumen expansion, or change in blood pressure. These labeling schemes are merely illustrative, and it should be understood that any suitable labeling scheme can be used. In some variations, training data is given multiple labels, such as labels associated with success of lesion removal and labels associated with side effects of a treatment.

At step 204, the training data 203 is used to train one or more machine learning models 205. The machine learning model(s) 205 can include a Bayesian model, a random forest model, a support vector machine learning model, a linear regression model, a non-linear regression model, a multivariate regression machine learning model, a robust machine learning model, a neural network model, a nearest neighbor machine learning model, a gradient boosting ensemble model, and/or a proportional hazards model. The machine learning model(s) can include one or more neural networks, such as a convolutional neural network (CNN), an artificial neural network (ANN), a recurrent neural network (RNN), and/or other type of neural network.

Training of the one or more machine learning models 205 results in one or more trained machine learning models 207 (often referred to as inference models). The one or more trained machine learning models 207 can be used for determining at least one parameter of a treatment of a target area of a body lumen with a lithotripsy catheter. The one or more trained machine learning models 207 can be incorporated into one or more applications executed by treatment optimization system 32. The one or more trained machine learning models can be executed remotely of treatment optimization system 32, such as at a server that is available to treatment optimization system 32. For example, treatment optimization system 32 may be local to a treatment room or facility and may communicate with a remote server executing one or more trained machine learning models for obtaining one or more treatment parameters.

Optionally, method 200 can include updating the machine learning model based on data associated with treatments conducted based on the output of the one or more machine learning models. At step 206, the trained machine learning model(s) 207 resulting from step 204 can be used to determine at least one parameter of a treatment that is predicted by the trained machine learning model(s) 207 to result in a favorable treatment outcome. The prediction can be, for example, a type or size of a catheter or one or more parameters of energy pulses for generating shock waves (e.g., voltage, pulse width, delivery by a relatively lower number of high amplitude pulses or delivery by a relatively higher number of low amplitude pulses). At step 208, the one or more parameters determined using the trained machine learning model(s) 207 are used for conducting a treatment. For example, the treatment can be conducted using the type of catheter determined by the trained machine learning model(s) 207 for use in the treatment. At step 210, treatment data is collected. The treatment data can be any of the types of data described above with respect to historical treatment data, including imaging data and/or operating settings of the energy pulse generator and/or fluid supply system. Method 200 then returns to step 202 in which the treatment data 209 collected at step 210 is labeled (e.g., based on one or more aspects of the treatment outcome) to produce additional training data 203. Step 204 is repeated using the additional training data to provide updated training of the trained machine learning model(s) 207. The updating of the machine learning model(s) 207 may be repeated as often as desired, including after every treatment, after a certain number of treatments, only a single time, multiple times, etc. Optionally, data from a treatment is stored locally or remotely for future use in training (or updating training) of one or more machine learning models 207. For example, the data from a treatment may be stored in a database to which data from future treatment assessments is to be added, and once a sufficient period of time has passed to enable assessment of the outcome of the treatment, the collected data is labeled and used to train one or more machine learning models 207.

The steps of method 200 can be performed by the same computing system or by multiple computing systems. For example, the data labeling of step 202 may be performed by a first computing system (or multiple first computing systems), and the machine learning of step 204 may be performed by a second computing system. The trained machine learning model(s) 207 may be installed on one or more computing systems that may be used by one or more treatment providers. For example, the trained machine learning model(s) 207 may be installed on treatment optimization system 32 of FIG. 1 at each treatment site employing system 100.

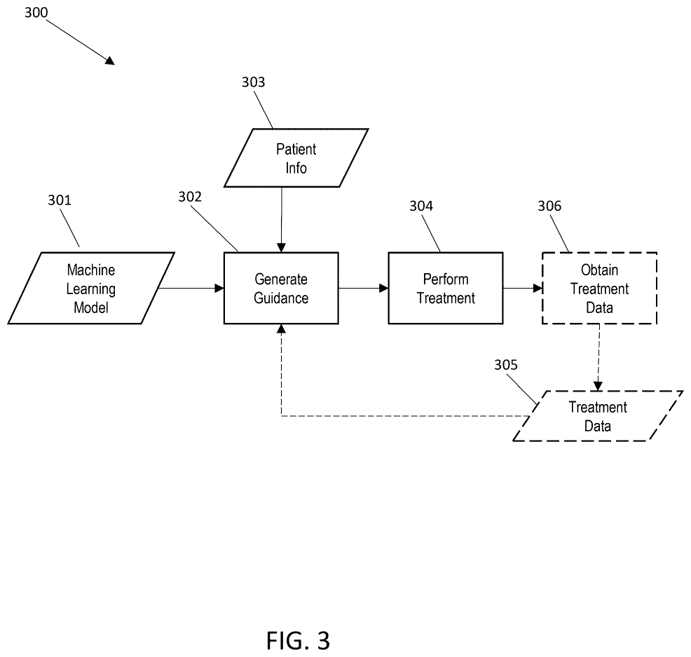

A machine learning model trained according to method 200 of FIG. 2 may be used to provide pre-treatment guidance to a treatment provider to guide the treatment provider in treating a lesion in a body lumen of a patient. FIG. 3 illustrates an exemplary method 300 for treating a target area of a body lumen using a lithotripsy catheter, such as catheter 10 of FIG. 1, in which a machine learning model is used to provide guidance for the treatment. At step 302, one or more machine learning model(s) 301 are used to generate guidance for treating a lesion in a body lumen (e.g., a partially or completed occluded body lumen) of a patient using a lithotripsy catheter. Any suitable computing system may be configured to use the machine learning model(s) 301 to generate the guidance. The machine learning model(s) 301 process patient-specific information 303 to generate the guidance. The patient-specific information 303 is information associated with the patient for whom the treatment will be performed. The patient-specific information can include measurements or other characteristics associated with the body lumen (diameter(s), location(s), type, etc.) and/or measurements or other characteristics associated with the lesion (lengths, widths, thicknesses, hardness, location, eccentricity, degree of plaque burden, etc.). Such measurements may be provided by medical personnel. For example, a surgeon or other medical personnel may determine any such measurements and enter the measurements into a patient record accessible to the machine learning model(s) 301. Additionally, or alternatively, measurements may be determined automatically from one or more scans of the body lumen. For example, treatment optimization system 32 may be configured to process scans of the body lumen to generate measurement associated with the body lumen and/or lesion. In some variations, the one or more scans are themselves included in patient-specific information 303 that is processed by the one or more machine learning models 301. Patient-specific information 303 may include patient demographic information (e.g., age, sex, body mass index, etc.), patient monitoring data (e.g., heart rate, blood pressure, etc.), pre-existing conditions or medications, or any other data associated with the patient that may be relevant to treating the lesion.

Step 302 may include the computing system receiving patient-specific information 303. The patient-specific information 303 may be received by the computing system in a number of different ways. Patient-specific information 303 may be loaded onto the computing system from a server system, such as a picture archiving and communication system (PACS), a hospital information system (HIS), or an electronic health record (EHR) system. The patient-specific information 303 may be loaded onto the computing system using a removable data storage device, such as a thumb drive. The patient-specific information may be entered into the computing system by medical personnel, such as via a user interface of the computing system. Patient-specific information such as imaging may be received from one or more imaging systems that are communicatively connected to the computing system.

Step 302 may include the computing system determining at least one parameter for a treatment of the at least partially occluded body lumen with the lithotripsy catheter. The at least one parameter can include any of the parameters discussed above or any combination of the parameters discussed above. For example, the at least one parameter can be a type of size of a catheter to be used for treating the lesion. The at least one parameter can be associated with operating a catheter, such as one or more parameters for operation of an energy pulse generator, such as pulse generator 28 of FIG. 1, and/or operation of a fluid supply system, such as fluid supply system 30 of FIG. 1. For example, the at least one parameter can be a mode that the treatment provider should select for operating the energy pulse generator and/or fluid supply system. The at least one parameter can be a specific setting of the energy pulse generator and/or fluid supply system, such as a setting of the energy pulse generator associated with pulse amplitude or pulse frequency, or a setting of the fluid supply system associated with the pressure of fluid within an enclosure of the catheter, such as enclosure 18 of FIG. 1.

The at least one parameter can be associated with steps of the treatment. For example, the at least one parameter can indicate the number of pulses (e.g., total number for the treatment, number per cycle, number of cycles per treatment, number of pulses for each shock wave emitter, etc.) that the treatment provider should use to treat a lesion or the amount of time of providing pulses the treatment provider should use to treat a lesion. The at least one parameter can indicate whether to pressurize or how much to pressurize an enclosure, such as an angioplasty balloon of the catheter. The at least one parameter can indicate whether multiple cycles of pulses should be performed, whether only certain shock wave emitters should be provided with energy pulses, and/or whether the catheter should be moved between cycles. The at least one parameter can indicate a suggested attribute of fluid used for the enclosure of the catheter, such as a suggested ratio of contrast to saline.

Generating guidance according to step 302 can include displaying guidance (e.g., displaying one or more parameters determined using at least one machine learning model) to a treatment provider on one or more displays. For example, one or more parameters determined by the computing system using the machine learning model(s) 301 and the patient-specific information 303 may be displayed as recommendations to a treatment provider on a display of the computing system. The guidance can be provided at any time, including prior to a treatment session (e.g., in a pre-operative planning phase), at the beginning of a treatment session (e.g., in the treatment location, with the patient prepped for treatment), during a treatment session, and/or after a treatment session.

Step 302 may be performed by any suitable computing system. Step 302 may be performed in the days or weeks before a treatment or may be performed immediately prior to the treatment. Step 302 may be performed by treatment optimization system 32 of system 100 of FIG. 1. Step 302 may be performed by a computing system used by a treatment provider or may be performed by a remote system that is communicatively coupled to a computing system used by a treatment provider. Step 302 may be performed by a mobile computing system, such as a tablet or smartphone, or by a computing system communicatively connected to a mobile computing system such that the guidance is displayed to the treatment provider on a display of the mobile computing system. The guidance provided according to step 302 may be displayed on one or more display devices of a computing system, such as one or more display devices in a treatment room, on a treatment cart, and/or a display of a mobile device of a treatment provider.

At step 304, the treatment is performed according to the guidance provided at step 302. For example, the recommended catheter type or size may be used, the recommended energy pulse mode or settings may be used, the fluid supply system mode or settings may be used, etc., to treat the lesion in the body lumen. The treatment may include generating one or more shock waves that disrupt the lesion. The treatment may include additional aspects, such as expanding a body lumen by inflating an angioplasty balloon of the catheter.

Method 300 may include providing guidance during the treatment based, at least in part, on treatment data gathered during the treatment. Method 300 may include optional step 306 in which treatment data 305 is obtained. The treatment data 305 can include operational data of the energy pulse generator and/or the fluid supply system. The treatment data 305 can include data associated with the catheter, such as data associated with sensors of the catheter (e.g., pressure and/or temperature sensors of the catheter). The treatment data 305 can include intra-operative imaging data, such as fluoroscopic imaging, IVUS imaging, and/or OCT imaging.

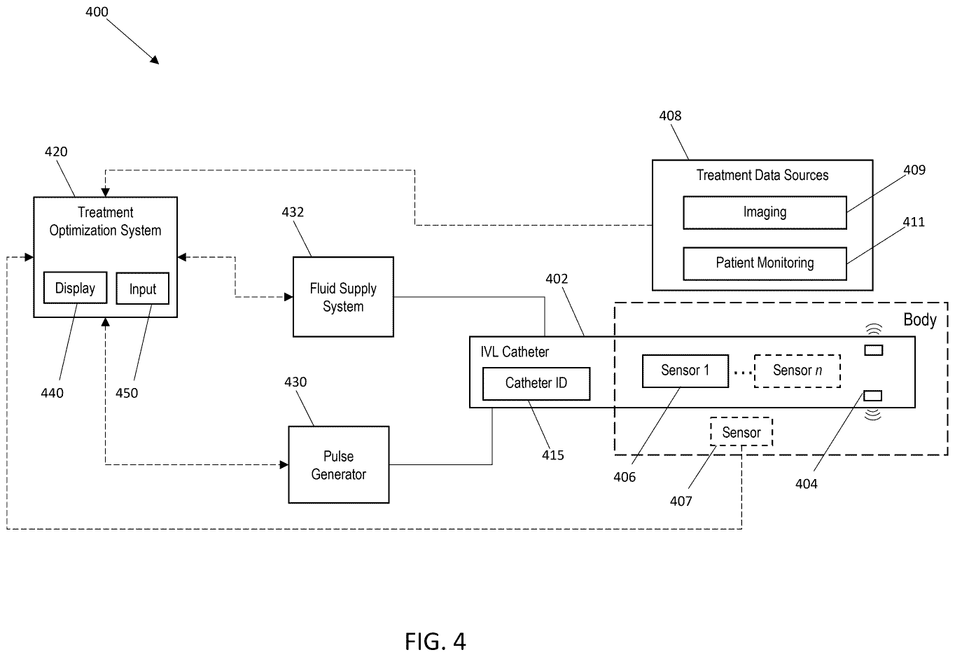

FIG. 4 illustrates an exemplary treatment system 400 configured for obtaining treatment data, such as for obtaining treatment data 305 in step 306 of method 300, and using the treatment data for providing updated guidance during lithotripsy treatment, such as in step 302 of method 300. System 400 includes an intravascular lithotripsy catheter 402 that includes one or more shock wave emitters 404 for emitting shock waves for treating a lesion, such as an occlusion, in a lumen of a body. The catheter 402 can be, for example, catheter 10 of FIG. 1.

System 400 includes a treatment optimization system 420 that can collect data associated with a treatment by the catheter 402. Treatment optimization system 420 may be communicatively connected to pulse generator 430 (e.g., pulse generator 28 of FIG. 1) and/or fluid supply system 432 (e.g., fluid supply system 30 of FIG. 1) or may be a feature of the energy pulse generator 430 or fluid supply system 432. Treatment optimization system 420 may receive data from the energy pulse generator 430 associated with operation of the energy pulse generator 430, such as a mode of the energy pulse generator 430, timing of pulse generation, and one or more parameters of pulse generation (e.g., amplitude and frequency). Similarly, treatment optimization system 420 may receive data from the fluid supply system 432 associated with operation of the fluid supply system 432, such as mode or pressure of fluid supplied to the catheter 402.

Treatment optimization system 420 may receive data from one or more sensors 406 of the catheter 402. The one or more sensors 406 may sense characteristics of operation of the catheter 402. For example, the one or more sensors 406 may include one or more pressure or temperature sensors that sense pressure and/or temperature within an enclosure (e.g., enclosure 18 of FIG. 1) of the catheter 402. The one or more sensors 406 may include one or more current or voltage sensors that sense current and/or voltage across one or more electrode pairs of one or more shock emitters. The data from the one or more sensors 406 may be received via the energy pulse generator 430 and/or the fluid supply system 432. For example, the one or more sensors 406 may be connected to circuitry within the energy pulse generator 430, and the energy pulse generator 430 may send sensor data to treatment optimization system 420. The one or more sensors 406 may be imaging sensors for imaging the body lumen from within. For example, the one or more sensors 406 may include one or more ultrasound sensors of an IVUS system or one or more optical imaging sensors of an OCT system.

Treatment optimization system 420 may receive other data associated with the catheter 402. For example, the catheter 402 may be configured to provide identification information, such as within a memory 415 of the catheter 402. The memory 415 may store information such as catheter type, model number, attributes, or any other suitable information. The memory 415 may be accessible to treatment optimization system 420 either directly (e.g., via a wireless communication connection) or via the energy pulse generator 430 and/or the fluid supply system 432.

Treatment optimization system 420 may receive data from one or more sensors 407 that are not integrated into the catheter 402. Such sensor(s) 407 may be located within the body or externally to the body. The sensor(s) 407 may include a pressure sensor located within a body lumen within which the catheter 402 is positioned or located externally of the body lumen within which the catheter 402 is positioned. The sensor(s) 407 may include a sensor configured to detect tissue properties (e.g., lesion properties), such as calcium hardness or density.

Treatment optimization system 420 may receive data from one or more other treatment data sources 408. The treatment data sources 408 may include one or more imaging data sources 409 (e.g., fluoroscopic imaging system, IVUS system, and/or OCT system) and/or one or more patient monitoring data sources 411 (e.g., patient vital sign monitoring systems).

Returning to FIG. 3, method 300 may return to step 302 in which the treatment data 305 obtained at step 306 (e.g., received by treatment optimization system 420) may be used to generate updated guidance during a treatment. Updated guidance may be generated by determining (by the computing system) an adjustment for at least one parameter for operating the catheter 402 using the machine learning model(s) 301 and the treatment data 305. The at least one parameter may be any of the parameters discussed herein. For example, the adjustment for the at least one parameter may be an increase in the amplitude of energy pulses to increase shock wave power or an increase in a pressure of fluid supplied to an angioplasty balloon of the catheter 402 to expand the lumen. The adjustment may be a change in distribution of energy pulses delivered to different shock wave emitters (e.g., delivering more energy pulses to more distal emitters than proximal emitters or vice versa). The adjustment may be a change in location of treatment and the updated guidance may be guidance on changing the location of the catheter according to the determined adjustment. The updated guidance (e.g., the parameter adjustment) may be provided to a treatment provider using any suitable user interface, such as display device 440 (e.g., display screen) of treatment optimization system 420 of FIG. 4.

Optionally, at least some of the treatment data 305 may be pre-processed before being provided to the machine learning model(s) 301. For example, imaging data may be processed to extract relevant information from the imaging data (e.g., vessel attributes) that is then provided to the machine learning model(s) 301. Treatment data 305 may be pre-processed using one or more other machine learning models, such as one or more machine learning models configured to identify features in imaging data.

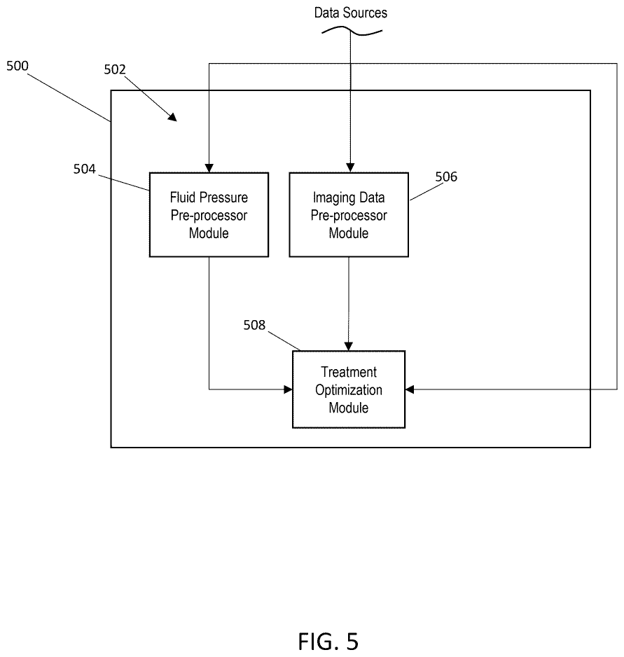

Pre-processing of treatment data 305 may be performed by the computing system generating the updated guidance. FIG. 5 is a functional block diagram of an exemplary computing system 500 configured to pre-process treatment data and generate updated treatment guidance. Computing system 500 may be used for treatment optimization system 420 of FIG. 4. Computing system 500 may include one or more treatment data pre-processing modules 502, which may receive treatment data from one or more external sources, such as from fluid supply system 432, pulse generator 430, or treatment data sources 408. The illustrated example includes a fluid pressure pre-processor module 504 and an imaging data pre-processor module 506. The fluid pressure pre-processor module 504 may receive data associated with pressure of fluid of a lithotripsy catheter, such as the pressure of fluid within an enclosure surrounding shock wave emitters of the catheter as sensed by a pressure sensor. The fluid pressure pre-processor module 504 may analyze the pressure data to extract relevant information, such as an average pressure in a given time period, a maximum pressure in a given time period, or any other pressure-related information. The imaging data pre-processor module 506 may process imaging data, such as angiographic images, to extract relevant information about a treatment. For example, the imaging data pre-processor module 506 may identify the body lumen receiving treatment in an angiographic image and may determine one or more characteristics of the body lumen or lesion being treated. The imaging data pre-processor module 506 may use one or more machine learning models and/or other image processing algorithms to extract relevant information from the imaging. For example, a first feature extractor machine learning model may be used to identify the body lumen in the imaging, a second feature extractor machine learning model may be used to identify the walls of the body lumen identified by the first feature extractor, and an algorithm configured to determined distances within the image based on distance between pixels and attributes of the image capture system (e.g., focal length, field of view, etc.) may determine the distance between the identified walls, thereby extracting the diameter of the body lumen.

The one or more treatment data pre-processing modules 502 may output relevant information (e.g., pressure values, lumen diameters, etc.) to a treatment optimization module 508 that uses a machine learning model (e.g., machine learning model(s) 301) to generate updated guidance during a treatment. Optionally, the treatment optimization module 508 may receive information directly from external sources. For example, the treatment optimization module 508 may receive energy pulse attributes (e.g., amplitude, frequency, pulse width, etc.) from an energy pulse generator (e.g., pulse generator 430 of FIG. 4).

Returning to FIG. 3, guidance may be generated at step 302 in response to a user input. For example, with reference to FIG. 4, treatment optimization system 420 may include one or more user inputs 450 that a user may use to request the treatment optimization system 420 to provide guidance. The one or more user inputs 450 can provided, additionally or alternatively, by the fluid supply system 432, the pulse generator 430, and/or the catheter 402 (e.g., located on a handle of the catheter 402). Exemplary user inputs 450 include a touchscreen, a mouse, a keyboard, a voice command system, and buttons. A user may request treatment guidance by selecting from a menu of guidance options. For example, prior to treatment, a user may request guidance on the type of catheter to use for treatment by selecting “catheter type guidance” or the like from a menu. Similarly, a user may request guidance on settings for use during a treatment (e.g., energy pulse generator settings) by selecting a “treatment settings guidance” or the like option from a menu.

In addition to, or alternatively to, providing guidance in response to a user request, treatment optimization system 420 may provide guidance automatically. For example, treatment optimization system 420 may determine that a pressure of fluid provided to the catheter should be increased (e.g., based on analysis of treatment data 305) and may automatically display guidance to the user.

A machine learning model trained according to method 200 of FIG. 2 may be used to provide automatic control one or more aspects of the operation of a lithotripsy catheter. For example, with reference to FIG. 1, treatment optimization system 32 may be configured to determine one or more parameters associated with the operation of catheter 10 and may send control commands (or otherwise control) fluid supply system 30 and/or pulse generator 28 according to the determined parameters, thereby automatically controlling treatment by the catheter 10.

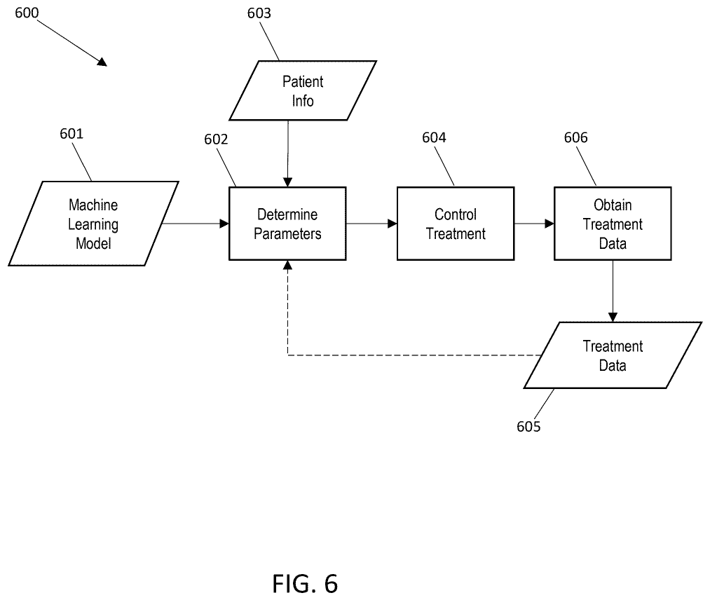

An exemplary method 600 for automatically controlling treatment of a target area of a body lumen using an intravascular lithotripsy catheter is illustrated in FIG. 6. Method 600 may be performed by a treatment system, such as system 100 of FIG. 1 or system 400 of FIG. 4. At step 602, patient-specific information 603 may be processed by one or more machine learning models 601 to determine one or more parameters for operating a lithotripsy catheter, such as catheter 10 of FIG. 1. Step 602 may be similar to step 302 of FIG. 3 except that the determined parameter(s) are used to control operation of the catheter, rather than to provide guidance. Optionally, methods 300 and 600 are combined such that guidance is provided to a treatment provider (e.g., guidance in selecting a suitable catheter type) and various aspects of operation of a catheter are automatically controlled. Step 602 may be performed by a suitable computing system, such as treatment optimization system 32 of FIG. 1 and treatment optimization system 420 of FIG. 4.

At step 604, the one or more parameters for operating a lithotripsy catheter determined in step 602 are used to automatically control one or more aspects of treatment by the lithotripsy catheter. For example, with reference to system 400 of FIG. 4, treatment optimization system 420 may determine one or more parameters for generating energy pulses and may provide one or more commands to the energy pulse generator 430 to generate energy pulses according to the one or more parameters. The one or more parameters for generating energy pulses may include, for example, pulse amplitude, pulse frequency, a number of pulses, a period of time for pulsing, etc. The energy pulse generator 430 may respond to the one or more commands by generating pulses according to the one or more parameters. Similarly, treatment optimization system 420 may determine one or more parameters for fluid supply to the catheter 402 and may provide one or more commands to the fluid supply system 432 to supply fluid to the catheter 402 according to the one or more parameters. The one or more parameters for fluid supply to the catheter 402 may include, for example, a pressure of fluid within an enclosure of the catheter (e.g., enclosure 18 of FIG. 1). The energy pulse generator 430 may respond to the one or more commands by supplying fluid to the catheter 402 according to the one or more parameters. Automatic control according to step 604 need not involve every parameter of operating of the catheter. Rather, as few as one parameter may be controlled, with other parameters being pre-set or manually controlled by a treatment provider.

Automatic control of operation of the lithotripsy catheter may be ongoing during delivery of the treatment. The ongoing automatic control may be based on treatment data obtained during the treatment. As such, method 600 may include step 606 in which treatment data 605 is obtained. Step 606 may be substantially the same as step 306 of method 300. Method 600 may return to step 602 in which the treatment data 605 is used to determine adjustments to one or more parameters for operating the lithotripsy catheter. For example, treatment optimization system 420 may determine from the treatment data 605 (using the machine learning model 601) that a lesion is not being sufficiently eroded, and treatment optimization system 420 may determine an adjustment to one or more parameters of the energy pulses provided to the catheter 402 to increase the effectiveness of the shock waves generated by the catheter. The determined adjustments to the energy pulse parameters may be used in step 604 to automatically adjust the generation of the energy pulses. As another example, treatment optimization system 420 may determine from the treatment data 605 (using the machine learning model 601) that a lesion has been being sufficiently eroded by shock wave treatment and that a pressure of fluid within an angioplasty balloon of the catheter should be increased to expand the balloon for expanding the body lumen. Treatment optimization system 420 may then send a command to the fluid supply system 432, at step 604, that causes the fluid supply system 432 to increase the pressure within the balloon of the catheter 402 to expand the catheter 402.

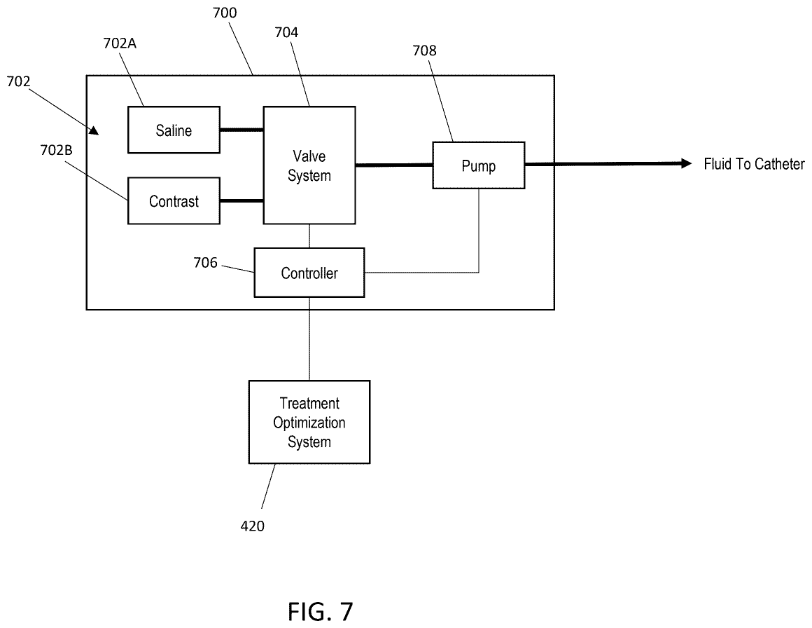

FIG. 7 illustrates an example arrangement for automatically controlling the operation of a fluid supply system 700 for supplying fluid to a lithotripsy catheter during lithotripsy treatment. Fluid supply system 700 may be configured to control properties of a fluid provided to the catheter, such as by controlling the relative proportion of different constituents of the fluid provided to the catheter. In the illustrated example, two constituents are shown—saline and contrast. However, system 700 can be configured for any number of constituents and can be configured to use constituents other than saline and contrast. In general, different mixtures of the constituents may result in different fluid properties that affect various aspects of use of the catheter. For example, a greater ratio of contrast to saline may result in higher density of the fluid, which may affect the formation and/or propagation of shock waves and/or cavitation bubbles. The change in the ratio of contrast to saline may affect the electrical conductivity of the fluid, which may affect the generation of a spark across electrodes. A ratio of contrast to saline may be automatically adjusted up or down (e.g., from a baseline, such as 50:50), such as to achieve different densities that result in different acoustic pressure applied to a treatment site, to achieve different electrical conductivities that result in different spark generation characteristics (e.g., more powerful sparks that provide greater acoustic pressure at the expense of greater electrode erosion, less powerful sparks that better preserve electrode material while providing sufficient acoustic energy, etc.), and/or to achieve different visibilities of the fluid. System 700 can control the relative proportion of different constituents of a fluid flow to a catheter for a specific treatment or a specific phase of a treatment and may do so automatically based on processing of patient-specific information by one or more machine learning models, according to the principles described herein. System 700 may be used for fluid supply system 432 of FIG. 4.

Fluid supply system 700 may include multiple fluid constituent sources 702. In the illustrated example, the fluid constituent sources include a saline source 702A and a contrast source 702B. The fluid constituent sources 702 can include one or more constituent reservoirs and/or one or more constituent supply lines. System 700 may include a valve system 704 for combining the different constituents into a single fluid flow for supplying to the catheter. One or more pumps 708 may control the pressure of the fluid supplied to the catheter. A controller 706 may control the valve system 704 to control the mixture of the different constituents and may control the pump 708 to control attributes of the fluid flow, such as flow rate and pressure.

Treatment optimization system 420 may be communicatively coupled to the controller 706 for providing instructions to the controller 706 for controlling aspects of the fluid flow provided to the catheter. Optionally, the functions of treatment optimization system 420 and controller 706 may be combined into the same component. For example, treatment optimization system 420 may include valve and/or pump control capabilities and may directly control the valve system 704 and/or the pump(s) 708. Alternatively, capabilities of treatment optimization system 420 may be embodied by the controller 706.

Treatment optimization system 420 may determine one or more parameters associated with operation of the fluid supply system 700 for treatment of a target treatment area by a catheter and may send instructions to the fluid supply system 700 (e.g., to controller 706) to operate according to the determined parameter(s). For example, with reference to method 600 of FIG. 6, at step 602, treatment optimization system 420 may determine that an optimal ratio of contrast to saline for a particular treatment is 50/50 and, at step 604, may send instructions to controller 706 to control the valve system 704 to provide a 50/50 ratio of contrast to saline.

Treatment optimization system 420 may control the fluid supply system 700 according to the determined parameters at any time, including before and/or during treatment. For example, treatment optimization system 420 may determine the ratio of constituents of the fluid to be supplied to the catheter at some point prior to the start of the treatment and may control fluid supply system 700 such that the desired fluid constituent ratio is set prior to the start of treatment. The set fluid constituent ratio may be maintained during the treatment.

Alternatively, the fluid constituent ratio may be changed at one or more times during treatment. Treatment optimization system 420 may determine a change to one or more parameters associated with operation of the fluid supply system 700, such as a change to the fluid constituent ratio, during the delivery of the treatment via the catheter and may control the fluid supply system 700 accordingly. For example, treatment optimization system 420 may determine that an occlusion is not being sufficiently treated (e.g., via machine learning-based analysis of treatment data, such as an angiogram) and may increase the ratio of contrast to saline to increase the density of the fluid surrounding the shock wave emitters (e.g., the fluid within enclosure 18 of catheter 10) and, thereby, increase the amplitude of shock waves delivered to the occlusion.

Controlling the fluid constituent ratio is merely one example of the control of the fluid supply system 700 by treatment optimization system 420. Another example of control of the fluid supply system 700 by treatment optimization system 420 is control of the fluid pressure supplied to the catheter. Treatment optimization system 420 may determine an optimal pressure of fluid supplied to the catheter and may send control commands to controller 706 to control the pump 708 to achieve the optimal pressure. Treatment optimization system 420 may determine the optimal fluid pressure dynamically throughout a treatment. For example, during a shock wave generating stage of a treatment, treatment optimization system 420 may control fluid supply system 700 to supply fluid at an optimal pressure for expanding enclosure (e.g., enclosure 18 of FIG. 1) surrounding shock wave emitters (e.g., shock wave emitter 16 of FIG. 1) such that the enclosure contacts and applies a suitable pressure to a vessel wall, and then during a vessel expansion phase, treatment optimization system 420 may control fluid supply system 700 to increase a pressure of the fluid to cause the enclosure to expand further, thereby expanding the vessel. Treatment optimization system 420 may automatically determine treatment stage transitions when pressure should transition from one level to another based on treatment data (e.g., obtained at step 606 of method 600). Additionally, or alternatively, a treatment provider may provide an input indicating a change in treatment stage that may be associated with pressure change. For example, a treatment provider may provide an input via user input 450 notifying treatment optimization system 420 that treatment of a lesion with shock waves is complete and vessel expansion should commence. In response, treatment optimization system 420 may determine the optimal fluid pressure for expansion of the enclosure (e.g., enclosure 18) and may control the fluid supply system 700 accordingly.

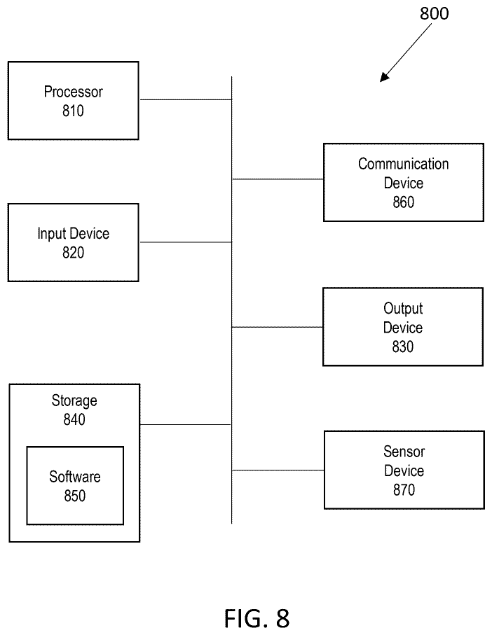

FIG. 8 illustrates an example of a computing system 800 that can be used for one or more components of system 100 of FIG. 1, such as fluid supply system 30, pulse generator 28, and/or treatment optimization system 32, one or more components of system 400 of FIG. 4, such as treatment optimization system 420, fluid supply system 432, pulse generator 430, and/or treatment data sources 408, one or more components of system 500 of FIG. 5, and one or more components of system 700 of FIG. 7, such as controller 706. System 800 can be a computer connected to a network, such as one or more networks of hospital, including a local area network within a room of a medical facility and a network linking different portions of the medical facility, or a wide-area network accessed through the internet or other means. System 800 can be a client or a server. System 800 can be any suitable type of processor-based system, such as a personal computer, workstation, server, handheld computing device (portable electronic device), such as a phone or tablet, or dedicated device. System 800 can include, for example, one or more of input device 820, output device 830, one or more processors 810, storage 840, and communication device 860. Input device 820 and output device 830 can generally correspond to those described above and can either be connectable or integrated with the computer.

Input device 820 can be any suitable device that provides input, such as a touch screen, keyboard or keypad, mouse, gesture recognition component of a virtual/augmented reality system, or voice-recognition device. Output device 830 can be or include any suitable device that provides output, such as a display, touch screen, haptics device, virtual/augmented reality display, or speaker.

Storage 840 can be any suitable device that provides storage, such as an electrical, magnetic, or optical memory including a RAM, cache, hard drive, removable storage disk, or other non-transitory computer-readable medium. Communication device 860 can include any suitable device capable of transmitting and receiving signals over a network, such as a network interface chip or device. The components of the computing system 800 can be connected in any suitable manner, such as via a physical bus or wirelessly.

Processor(s) 810 can be any suitable processor or combination of processors, including any of, or any combination of, a central processing unit (CPU), field programmable gate array (FPGA), and application-specific integrated circuit (ASIC). Software 850, which can be stored in storage 840 and executed by one or more processors 810, can include, for example, the programming that embodies the functionality or portions of the functionality of the present disclosure (e.g., as embodied in the devices as described above), such as programming for performing one or more steps of method 200, method 300, and/or method 600.

Software 850 can also be stored and/or transported within any non-transitory computer-readable storage medium for use by or in connection with an instruction execution system, apparatus, or device, such as those described above, that can fetch instructions associated with the software from the instruction execution system, apparatus, or device and execute the instructions. In the context of this disclosure, a computer-readable storage medium can be any medium, such as storage 840, that can contain or store programming for use by or in connection with an instruction execution system, apparatus, or device.

Software 850 can also be propagated within any transport medium for use by or in connection with an instruction execution system, apparatus, or device, such as those described above, that can fetch instructions associated with the software from the instruction execution system, apparatus, or device and execute the instructions. In the context of this disclosure, a transport medium can be any medium that can communicate, propagate, or transport programming for use by or in connection with an instruction execution system, apparatus, or device. The transport computer-readable medium can include, but is not limited to, an electronic, magnetic, optical, electromagnetic, or infrared wired or wireless propagation medium.

System 800 may include a sensor device 870 that provides sensor data for processing by processor 810. Sensor device 870 may be any of the sensors described herein. Sensor device 870, in some embodiments, may be an imaging sensor that provides imaging data, for a lesion being treated. In some embodiments, sensor device 870 may be a voltage sensor, a current sensor, a pressure sensor, a temperature sensor, or an optical sensor for providing data about a state of the catheter or a lesion.

System 800 may be connected to a network, which can be any suitable type of interconnected communication system. The network can implement any suitable communications protocol and can be secured by any suitable security protocol. The network can comprise network links of any suitable arrangement that can implement the transmission and reception of network signals, such as wireless network connections, T1 or T3 lines, cable networks, DSL, or telephone lines.

System 800 can implement any operating system suitable for operating on the network. Software 850 can be written in any suitable programming language, such as C, C++, Java, or Python. In various examples, application software embodying the functionality of the present disclosure can be deployed in different configurations, such as in a client/server arrangement or through a Web browser as a Web-based application or Web service.

Although the shock wave emitters and catheter devices described herein have been discussed primarily in the context of treating coronary occlusions, such as lesions in vasculature, the shock wave emitters and catheters herein can be used for a variety of occlusions, such as occlusions in the peripheral vasculature (e.g., above-the-knee, below-the-knee, iliac, carotid, etc.), or other hardened lesions, such as calcified heat valves. For further examples, various embodiments may be used for treating soft tissues, such as cancer and tumors (i.e., non-thermal ablation methods), blood clots, fibroids, cysts, organs, scar and fibrotic tissue removal, or other tissue destruction and removal. Electrode assembly and catheter designs could also be used for neurostimulation treatments, targeted drug delivery, treatments of tumors in body lumens (e.g., tumors in blood vessels, the esophagus, intestines, stomach, or vagina), wound treatment, non-surgical removal, and destruction of tissue, or used in place of thermal treatments or cauterization for venous insufficiency and fallopian ligation (i.e., for permanent female contraception).

The electrode assemblies and catheters described herein may be used for tissue engineering methods, for instance, for mechanical tissue decellularization to create a bioactive scaffold in which new cells (e.g., exogenous or endogenous cells) can replace the old cells; introducing porosity to a site to improve cellular retention, cellular infiltration/migration, and diffusion of nutrients; and signaling molecules to promote angiogenesis, cellular proliferation, and tissue regeneration similar to cell replacement therapy. Such tissue engineering methods may be useful for treating ischemic heart disease, fibrotic liver, fibrotic bowel, and traumatic spinal cord injury (SCI). For instance, for the treatment of spinal cord injury, the devices and assemblies described herein could facilitate the removal of scarred spinal cord tissue, which acts like a barrier for neuronal reconnection, before the injection of an anti-inflammatory hydrogel loaded with lentivirus to genetically engineer the spinal cord neurons to regenerate.

It should be noted that the elements and features of the example catheters illustrated throughout this specification and drawings may be rearranged, recombined, and modified without departing from the present invention. For instance, while this specification and drawings describe and illustrate catheters having several example balloon designs, the present disclosure is intended to include catheters having a variety of balloon configurations. The number, placement, and spacing of the shock wave emitters can be modified without departing from the subject invention. Further, the number, placement, and spacing of balloons of catheters can be modified without departing from the subject invention.

It should be understood that the foregoing is only illustrative of the principles of the invention, and that various modifications, alterations, and combinations can be made by those skilled in the art without departing from the scope and spirit of the invention. Any of the variations of the various catheters disclosed herein can include features described by any other catheters or combination of catheters herein. Furthermore, any of the methods can be used with any of the catheters disclosed. Accordingly, it is not intended that the invention be limited, except as by the appended claims.

1. A method for treating a target area of a body lumen using an intravascular lithotripsy catheter, the method comprising:
receiving, at a computing system, patient-specific information for a patient that has the at least partially occluded body lumen;
determining, by the computing system, at least one parameter of a treatment of the at least partially occluded body lumen with the intravascular lithotripsy catheter, wherein the at least one parameter is determined by processing the patient-specific information with at least one machine learning model; and
displaying, by the computing system, guidance for treating the at least partially occluded body lumen with the intravascular lithotripsy catheter based on the at least one parameter, wherein treating the at least partially occluded body lumen with the intravascular lithotripsy catheter comprises the intravascular lithotripsy catheter generating at least one shock wave.

2. The method of claim 1, wherein the patient-specific information comprises imaging data capturing the at least partially occluded body lumen.

3. The method of claim 1, wherein the patient-specific information comprises measurements of the body lumen and/or an occlusion in the body lumen.

4. The method of claim 1, wherein the at least one parameter comprises a type of the intravascular lithotripsy catheter or a size of the intravascular lithotripsy catheter.

5. The method of claim 1, wherein the at least one parameter comprises at least one parameter for operating the intravascular lithotripsy catheter.

6. The method of claim 5, wherein the at least one parameter for operating the intravascular lithotripsy catheter comprises a fluid filling pressure of an enclosure of the intravascular lithotripsy catheter, or at least one parameter of at least one pulse of energy provided to the intravascular lithotripsy catheter for generating shock waves.

7. The method of claim 6, wherein the at least one parameter of the at least one pulse of energy comprises a pulse frequency, a pulse amplitude, and a total number of pulses.

8. The method of claim 6, wherein the at least one parameter comprises a number of cycles of pulses.

9. The method of claim 6, wherein the at least one pulse of energy is at least one electrical pulse or at least one laser pulse.

10. A system for treating a target area of a body lumen using an intravascular lithotripsy catheter, the system comprising one or more processors and memory storing one or more programs for execution by the one or more processors, the one or more programs including instructions that when executed by the one or more processors cause the system to perform the method of claim 1.

11. A method for treating a target area of a body lumen using an intravascular lithotripsy catheter, the method comprising:
receiving, at a computing system, data generated during treatment of the at least partially occluded body lumen by the intravascular lithotripsy catheter;
determining, by the computing system, at least one parameter for operating the intravascular lithotripsy catheter by processing the data generated during the treatment of the at least partially occluded body lumen with at least one machine learning model; and
providing guidance to a treatment provider for operating the intravascular lithotripsy catheter in accordance with the at least one parameter for operating the intravascular lithotripsy catheter.

12. The method of claim 11, wherein the data generated during the treatment of the at least partially occluded body lumen by the intravascular lithotripsy catheter comprises imaging data capturing the at least partially occluded body lumen.

13. The method of claim 11, wherein the data generated during the treatment of the at least partially occluded body lumen by the intravascular lithotripsy catheter comprises measurements of the body lumen and/or an occlusion in the body lumen.

14. The method of claim 11, wherein the at least one parameter comprises at least one parameter for operating the intravascular lithotripsy catheter.

15. The method of claim 14, wherein the at least one parameter for operating the intravascular lithotripsy catheter comprises a fluid filling pressure of an enclosure of the intravascular lithotripsy catheter, or at least one parameter of at least one pulse of energy provided to the intravascular lithotripsy catheter for generating shock waves.

16. The method of claim 15, wherein the at least one parameter of the at least one pulse of energy comprises a pulse frequency, a pulse amplitude, and a total number of pulses.

17. The method of claim 16, wherein the at least one parameter comprises a number of cycles of pulses.

18. The method of claim 16, wherein the at least one pulse of energy is at least one electrical pulse or at least one laser pulse.

19. The method of claim 11, wherein the at least one parameter for operating the intravascular lithotripsy catheter is determined by processing patient-specific data received by the computing system prior to the treatment.

20. A system for treating a target area of a body lumen using an intravascular lithotripsy catheter, the system comprising one or more processors and memory storing one or more programs for execution by the one or more processors, the one or more programs including instructions that when executed by the one or more processors cause the system to perform the method of claim 11.

21. A method for treating a target area of a body lumen using an intravascular lithotripsy catheter, the method comprising:
receiving, at a computing system, data generated during treatment of the at least partially occluded body lumen by the intravascular lithotripsy catheter;
determining, by the computing system, at least one parameter for operating the intravascular lithotripsy catheter by processing the data associated with treatment of the occlusion with at least one machine learning model; and
automatically controlling treatment by the intravascular lithotripsy catheter according to the at least one parameter to treat the occlusion.

22. The method of claim 21, wherein the at least one parameter comprises a fluid filling pressure of an enclosure of the intravascular lithotripsy catheter or at least one parameter of at least one pulse of energy provided to the intravascular lithotripsy catheter for generating shock waves.

23. The method of claim 22, wherein the at least one parameter of the at least one pulse of energy comprises a pulse frequency, a pulse amplitude, and a total number of pulses.

24. The method of claim 21, wherein the data generated during the treatment of the occlusion comprises imaging data capturing the at least partially occluded body lumen.

25. The method of claim 21, wherein automatically controlling treatment by the intravascular lithotripsy catheter according to the at least one parameter comprises automatically controlling, by the computing system, an energy pulse generator that provides energy pulses to the intravascular lithotripsy catheter for generating the at least one shock wave.

26. The method of claim 21, wherein automatically controlling treatment by the intravascular lithotripsy catheter according to the at least one parameter comprises automatically controlling, by the computing system, a pump to control a fluid pressure within a fluid enclosure of the intravascular lithotripsy catheter.

27. The method of claim 21, wherein the intravascular lithotripsy catheter comprises a fluid filled enclosure within which the at least one shock wave is generated, and wherein the at least one parameter comprises a proportion of contrast in a fluid that fills the fluid filled enclosure.

28. The method of claim 27, wherein automatically controlling treatment by the intravascular lithotripsy catheter according to the at least one parameter comprises automatically controlling, by the computing system, at least one valve for controlling the proportion of contrast in the fluid.

29. The method of claim 21, wherein the at least one parameter for operating the intravascular lithotripsy catheter is determined by processing patient-specific data received by the computing system prior to the treatment.

30. A system for treating a target area of a body lumen using an intravascular lithotripsy catheter, the system comprising one or more processors and memory storing one or more programs for execution by the one or more processors, the one or more programs including instructions that when executed by the one or more processors cause the system to perform the method of claim 21.
